(19) United States
(12) Patent Application Publication
(10) Pub. No.: US 2020/0230236 A1
(43) Pub. Date: Jul. 23, 2020

(54) COMBINATION FOR T-CELL IMMUNOTHERAPY AND USE THEREOF

(71) Applicant: MANYSMART THERAPEUTICS, INC., TAIPEI CITY (TW)

(72) Inventor: HSIN-YI HUANG, TAIPEI CITY (TW)

(73) Assignee: MANYSMART THERAPEUTICS, INC., TAIPEI CITY (TW)

(21) Appl. No.: 16/652,901

(22) PCT Filed: Sep. 21, 2018
(86) PCT No.: PCT/US2018/052183

§ 371 Date: Apr. 1, 2020

(30) Foreign Application Priority Data
Oct. 3, 2017 (TW) 106134195

(51) Int. Cl. A61K 39/395; A61K 35/17; A61P 35/02

(52) U.S. Cl. CPC A61K 39/3955; A61P 35/02; A61K 35/17

(57) ABSTRACT

Combinations including a bispecific T cell activating antigen binding molecule and effector memory γ9δ2 T cells. Also methods of using the combinations in treating diseases such as cancers and infectious diseases.

### FIELD OF THE INVENTION

The invention relates to a combination comprising T cells and a bispecific T cell activating antigen binding molecule, and a method of using said combination in immunotherapy, such as in the treatment of cancers.

### BACKGROUND

In order to treat diseases in clinic, selection and destruction of individual cells or a specific type of cells in a subject is often desirable. For example, in cancer treatment, a primary goal is to specifically destroy tumor cells while leaving healthy cells and tissues intact and undamaged.

To achieve the above goal, many different bispecific antibodies that can be used in T-cell mediated immunotherapy have been developed. Bispecific T cell Engager (BiTE) is one that has been well studied and shows potential in clinic (Holliger et al., Prot Eng 9, 299-305 (1996); Kipriyanov et al., J Mol Biol, 293, 41-66 (1999); Nagorsen and Bäuerle, Exp Cell Res, 317, 1255-1260 (2011), and Sheridan, Nature Biotechnology, 34:1215-1217 (2016)). BiTEs are tandem scFv molecules, in which two scFv molecules are fused by a flexible linker. Blinatumomab (BLINCYTO®) is a BiTE formed by linking two single-chained antibodies each against CD19 and CD3 (U.S. Pat. No. 7,635,472 B2), which can be used to treat refractory B-cell acute lymphocytic leukemia (ALL) through T cell mediated immunotherapy. Clinical application of BiTE is, however, by no means trivial, and involves a number of challenges, such as efficacy, toxicity, applicability and production of the antibodies. Moreover, the therapeutic effect of BiTE may be greatly limited when the number and function of T cells in a patient are insufficient (as a result of, for example, subjection to chemotherapy or bone marrow transplantation), or when there are other diseases present. For instance, blinatumomab showed poor therapeutic effect when used to treat ALL patients who had extramedual diseases (Aldoss I et al., Am J Hematol., 92, 858-865 (2017)).

Wang and Riviere (Cancer Gene Ther., 22, 85-94 (2015) disclose that T cells can be used as a therapeutic agent against cancers by expanding tumor-specific cells ex vivo and reinfusing these cells into a patient. Chimeric antigen receptors (CARs) are recombinant receptors specific to an antigen. CARs can redirect the specificity and efficacy of T cells and other immune cells. The general premise for their use in cancer immunotherapy is to rapidly generate tumor-targeting T cells, bypassing the obstacles of active immunization. Once a CAR is expressed in cells, the CAR-modified T cells would become tumor-targeting T cells, which can exert both immediate and long-term effects in a patient. However, the applicability of CARs is limited by the necessity for the T cells to originate from the patient him/herself. In addition, CARs have to first be transduced into T cells to form CAR T cells, and such CAR T cells take a long time to expand before they can be autologously transplanted back into the patient's body.

Therefore, there is still need for technology and methods for improving therapeutic effect in T cell immunotherapy.

### SUMMARY

The present invention surprisingly found that bispecific T cell activating antigen binding molecules can be administered to patients together with effector memory γ9δ2 T cells, and that such administration can synergistically enhance the therapeutic effect of immunotherapy.

Accordingly, one aspect of the invention is to provide a method for treating a disease in a subject, comprising administering to said subject a therapeutically effective amount of a bispecific T cell activating antigen binding molecule and a therapeutically effective amount of effector memory γ9δ2 T cells.

Another aspect of the invention is to provide a combination, comprising a therapeutically effective amount of a bispecific T cell activating antigen binding molecule and a therapeutically effective amount of effector memory γ9δ2 T cells.

Another aspect of the invention is to provide the use of effector memory γ9δ2 T cells in the manufacture of a medicament for treating a disease in combination with a bispecific T cell activating antigen binding molecule.

Another aspect of the invention is to provide a kit, comprising a bispecific T cell activating antigen binding molecule and effector memory γ9δ2 T cells.

All publications, patent applications, patents and other references mentioned herein are incorporated by reference in their entirety.

Advantages and features of the present invention herein disclosed will become apparent from the following description, the accompanying drawings, and the claims. Furthermore, it is to be understood that the features of the various embodiments described herein are not mutually exclusive and can exist in various combinations and permutations.

### BRIEF DESCRIPTION OF THE DRAWINGS

FIG. 1 shows the ratio of δ2 T cells after being stimulated and cultured with interleukin-2 (IL-2) and Zoledronic acid for 14 days.

FIG. 2A shows that δ2 cells are a type of T cells; and FIG. 2B shows that the ratio of isolated γ9δ2 cells may be up to 98%.

FIG. 3A shows that the isolated γ9δ2 T cells are all CD27(−) and CD45RA(−) effector memory γ9δ2 T cells; and FIG. 3B shows that the isolated γ9δ2 T cells do not express PD-1.

FIG. 4A shows the effect on killing Raji, RPMI-8226, VAL and Daudi cell lines with treatment of γ9δ2 T cells or BLINCYTO® alone or in combination. FIG. 4B shows the effect on killing Raji and RPMI-8226 cell lines with treatment of γ9δ2 T cells or BCMA-BiTE alone or in combination.

FIG. 5A shows the viability of VAL blood cancer cells in the bone marrow of NOG mice that were not treated with γ9δ2 T cells and BLINCYTO®, and FIG. 5B shows the viability of VAL blood cancer cells in the bone marrow of NOG mice that were treated with the combination of γ9δ2 T cells and BLINCYTO®.

FIG. 6A shows the survival curve of the VAL blood cancer cells in NOG mice (without PBMC transplantation) simulating patients having early recurrence after receiving bone marrow transplantation; and FIG. 6B shows the relative survival days (p<0.01) of the VAL blood cancer cells in NOG mice (without PBMC transplantation) simulating patients having early recurrence after receiving bone marrow transplantation.

FIG. 7A shows the survival curve of the VAL blood cancer cells in NOG mice (with PBMC transplantation) simulating patients having early recurrence without receiving bone marrow transplantation; and FIG. 7B shows the relative survival days (p<0.01) of the VAL blood cancer cells in NOG mice (with PBMC transplantation) simulating patients having early recurrence without receiving bone marrow transplantation.

FIG. 8 shows the effect of the combination therapy with γ9δ2 T cells and BLINCYTO®, and monotherapy with BLINCYTO® on reducing extramedual cancer cell growth.

### DETAILED DESCRIPTION

The invention can be better understood by reference to the different embodiments, examples and tables, as well as the detailed description related thereto as set forth below. Unless defined otherwise, all the terms including technical and scientific terms used herein have the same meanings as commonly understood by one of ordinary skill in the art to which this invention belongs. It should be further understood that, as defined in commonly used dictionaries, the terms should be interpreted as having the same meanings as in articles of related art. In addition, unless otherwise defined in the present disclosure, the terms are not to be interpreted as idealistic or overly rigid. It should be further understood that the terms used in the present disclosure are for describing certain embodiments and not for limiting the invention.

It should be noted that as used herein and in the appended claims, the singular forms “a,” “an” and “the” include plural reference unless the context clearly dictates otherwise. Therefore, unless deemed necessary according to the context, singular terms should include plural reference.

Often, ranges are expressed herein as from “about” one particular value and/or to “about” another particular value. When such a range is expressed, an embodiment includes the range from the one particular value and/or to the other particular value. Similarly, when values are expressed as approximations, by use of the word “about,” it will be understood that the particular value forms another embodiment. It will be further understood that the endpoints of each of the ranges are significant both in relation to and independently of the other endpoint. As used herein the term “about” refers to ±20%, preferably ±10%, and even more preferably ±5%.

As used herein, unless otherwise indicated, terms such as “contain (containing),” “include (including),” and the like mean “to comprise (comprising).”

### Definitions

As used herein, the term “antigen binding molecule” refers in its broadest sense to a molecule that specifically binds an antigenic determinant. Examples of antigen binding molecules are immunoglobulins and derivatives thereof, such as molecules of full length or complete structure of an immunoglobulin and/or its functional fragments and/or variable heavy chain (VH) and/or variable light chain (VL) domains derived from antibodies. Therefore, antigen binding molecules can bind to a specific target or antigen. Antigen binding molecules have at least three light chain CDRs (i.e. CDR1, CDR2 and CDR3 of VL region), and/or three heavy chain CDRs (i.e. CDR1, CDR2 and CDR3 of VH region), and preferably have all six CDRs present. Antibody based antigen binding molecules include, for example, monoclonal, recombinant, chimeric, humanized and human antibodies.

Preferably, the CDRs are comprised in the VL and VH domains of the antibody, both of which, however, are not necessarily concurrently present in an antigen binding molecule. For example, an Fd fragment has two VH regions and generally retains certain antigen binding function of the complete antigen-binding domain. Examples of other formats of antibody fragments, variations or binding domains include: (1) Fab fragment, which is a monovalent fragment having VL, VH, CL and CH1 domains; (2) F(ab′)₂ fragment, which is a bivalent fragment having two Fab fragments linked by the disulfide bridge of the hinge region; (3) Fd fragment, which has two VH and CH1 domains; (4) Fv fragment, which has the VL and VH domains of one arm of an antibody; (5) dAb fragment, which has VH domain; (6) isolated complementarity determining regions (CDR); and (7) single chain Fv (scFV).

The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments, the bispecific antigen binding molecule is capable of binding two antigenic determinants at the same time, particularly to two antigenic determinants expressed on two distinct cells.

As used herein, the term “antigen binding moiety” means the polypeptide molecule that specifically binds to an antigenic determinant. In one embodiment, an antigen binding moiety is able to direct the entity to which it is attached (e.g. a second antigen binding moiety) to a target site, for example, to a specific type of tumor cell or tumor stroma bearing the antigenic determinant. In another embodiment an antigen binding moiety is able to activate signaling through its target antigen, for example a T cell receptor complex antigen. Antigen binding moieties include antibodies and fragments thereof. Particular antigen binding moieties include antigen binding domains of an antibody, which comprise the heavy chain variable region and the light chain variable region of the antibody. In certain embodiments, the antigen binding moieties may comprise antibody constant regions known in the art. Useful heavy chain constant regions include any of the five isotypes: α, δ, ε, γ, or μ. Useful light chain constant regions include any of the two isotypes: κ and λ.

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein (e.g. CD3) can be any native form of the proteins from any vertebrate source, including mammals such as primates (e.g. humans) and rodents (e.g. mice and rats), unless otherwise indicated. In a particular embodiment, the antigen is a human protein. An exemplary human protein useful as antigen is CD3, particularly the epsilon subunit of CD3, or CD19, also known as B-lymphocyte antigen CD19 or B-lymphocyte surface antigen B4. In certain embodiments, the bispecific T cell activating antigen binding molecule of the invention binds to an epitope of CD3 and/or CD19.

By “specific binding” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique and traditional binding assays. In certain embodiments, an antigen binding moiety that binds to the antigen, or an antigen binding molecule comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸ M or less, e.g. from 10⁻⁸M to 10⁻¹³M, e.g., from 10⁻⁹ M to 10⁻¹³ M).

An “activating T cell antigen” as used herein refers to an antigenic determinant expressed on the surface of a T lymphocyte, particularly an effector memory γ9δ2 T cell, which is capable of inducing T cell activation upon interaction with an antigen binding molecule. Specifically, interaction of an antigen binding molecule with an activating T cell antigen may induce T cell activation by triggering the signaling cascade of the T cell receptor complex. In a particular embodiment, the activating T cell antigen is CD3, particularly the epsilon subunit of CD3.

“T cell activation” as used herein refers to one or more cellular responses of a T lymphocyte, particularly an effector memory γ9δ2 T cell, selected from: proliferation, differentiation, cytokine secretion, cytotoxic effector molecule release, cytotoxic activity, and expression of activation markers. In certain embodiments, bispecific T cell activating antigen binding molecules of the invention are capable of inducing T cell activation.

A “target cell antigen” as used herein refers to an antigenic determinant presented on the surface of a target cell, for example a cell in a tumor such as a cancer cell or a cell of the tumor stroma. In a particular embodiment, the target cell antigen is CD19, particularly human CD19.

“γ9δ2 T cells” are a type of naturally occurring T cells present in peripheral blood which only account for 1-5% of all T cells in peripheral blood. γ9δ2 T cells can distinguish normal and abnormal cells by recognizing the isopentenyl pyrophosphate (IPP) molecules on cell surface. γ9δ2 T cells do not require human leukocyte antigen (HLA) for recognizing abnormal cells, and thus, the use thereof is irrelevant to HLA. This characteristic allows the use of γ9δ2 T cells in xenogenic subject without causing graft versus host disease.

As used herein, “effector memory γ9δ2 T cells” refer to CD27(−) and CD45RA(−) cells. In one certain embodiment, effector memory γ9δ2 T cells do not express programmed death 1 (PD1 or PD-1) immune checkpoint molecules.

As used herein, the term “combined administration” or the like means administration of chosen agents to a single patient simultaneously or separately via the same or different route of administration.

An “effective amount” of an agent refers to the amount that is necessary to result in a physiological change in the cell or tissue to which it is administered.

A “therapeutically effective amount” of an agent, e.g., a pharmaceutical composition, refers to an amount effective, at necessary dosages and time periods, to achieve the desired therapeutic or prophylactic result. For example, a therapeutically effective amount of an agent may eliminate, decrease, delay, minimize or prevent adverse effects of a disease.

The term “enhance (or enhancing)” means increasing or extending the efficacy or duration of the desired effect. As an example, to “enhance” an agent is to increase or extend the efficacy or duration of the effect of the agent on treating a disease, disorder or condition. As used herein, “enhancing effective amount” means the amount sufficient to enhance the effect of an agent in treating a disease, disorder or condition. When used in patients, the effective amount is determined based on the severity of the disease, disorder or condition, treatment priors, health condition and drug response of the patient, and the discretion of the attending physician.

A “patient” or “subject” is a mammal, which includes, but is not limited to, domesticated animals (e.g. cows, sheep, cats, dogs, and horses), primates (e.g. humans and non-human primates such as monkeys), rabbits, and rodents (e.g. mice and rats). Particularly, the patient or subject is a human.

The “subject in need” or “in need of treatment” refers to those already with the disorder as well as those in which the disorder is to be prevented.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the combination of the present invention can be used to delay development of a disease or to slow the progression of a disease.

“Cancer recurrence,” “cancer relapse” and “recurrent or refractory disease” are used interchangeably herein to refer to the reappearance of cancer after treatment, and include the reappearance of cancer in the organ of origin as well as distant recurrence in which cancer reappears outside the organ of origin.

The term “autologous” and its grammatical equivalents as used herein refer to originating from the same being. For example, a sample (e.g., cells, tissues, or organs) can be removed, processed, and given back to the same subject (e.g., patient) at a later time. An autologous process is distinguished from an allogenic process where the donor and the recipient are different subjects.

The term “xenogenic” and its grammatical equivalents as used herein refer to originating from different species. For example, a sample (e.g., cells, tissues, or organs) can be removed from a donor and processed, and then later transplanted into a recipient of different species.

The term “allogenic” and its grammatical equivalents as used herein refer to the recipient and donor being different subjects of the same species. For example, a sample (e.g., cells, tissues, or organs) can be removed from a donor of one species and processed, and then later transplanted into a different recipient of the same species.

The phrase “pharmaceutically or pharmacologically acceptable” means that a molecular entity and composition when used in a dosage and concentration is generally nontoxic to the recipient. That is, when administered to an animal, such as human when appropriate, the molecular entity and composition does not cause detrimental, allergic or other adverse reactions.

The term “package insert” is used to refer to instructions customarily included in commercial packages of therapeutic products that contain information about the indications, usage, dosage, administration, combination therapy, contraindications and/or warnings concerning the use of such therapeutic products.

### Combination Therapy and Use

The invention surprisingly found that when effector memory γ9δ2 T cells and a bispecific T cell activating antigen binding molecule are administered to a patient in combination, they can synergistically enhance the therapeutic effect of bispecific T cell activating antigen binding molecule mediated T cell immunotherapy. Accordingly, one of the embodiments of the invention is a combination therapy of administering to a subject in need thereof a therapeutically effective amount of a bispecific T cell activation antigen binding molecule and a therapeutically effective amount of effector memory γ9δ2 T cells, which can be used for treating diseases such as cancers, infectious diseases and/or immune disorders in the subject in need thereof. In another embodiment, the invention provides the use of effector memory γ9δ2 T cells in the manufacture of a medicament for treating diseases in combination with a bispecific T cell activating antigen binding molecule.

In one embodiment, the effector memory γ9δ2 T cells are administered simultaneously with, before or after the administration of the bispecific T cell activating antigen binding molecule. Accordingly, a bispecific T cell activating antigen binding molecule and effector memory γ9δ2 T cells may be administered simultaneously, sequentially or intermittently. In one embodiment, the effector memory γ9δ2 T cells and bispecific T cell activating antigen binding molecule may be administered simultaneously as a single active ingredient or two individual compositions, or administered sequentially as two individual compositions.

In one embodiment, the bispecific T cell activating antigen binding molecule comprises:

(a) a first antigen binding moiety that specifically binds to a first antigen, wherein the first antigen is CD3, which activates T cell; and

(b) a second antigen binding moiety that specifically binds to a second antigen, wherein the second antigen is selected from the group consisting of a tumor cell neo-antigen, a tumor neo-epitope, a tumor-specific antigen, a tumor associated antigen, a tissue-specific antigen, a bacterial antigen, a viral antigen, a yeast antigen, a fungal antigen, a protozoan antigen, and a parasite antigen.

In a preferred embodiment, the second antigen may include, but is not limited to, CD19, CD20, CD22, CD31, CD32B, CD33, CD34, CD40, CD117, CD123, fibroblast-activating protein (FAP), fibroblast growth factor receptor 1 (FGFR1), B-cell maturation antigen (BCMA), carcinoembryonic antigen (CEA), endothelial growth factor receptor, glycoprotein A33 antigen (gpA33), human epidermal growth factor receptor 1 (HER1), human epidermal growth factor receptor 2 (HER2/neu), human epidermal growth factor receptor 3 (HER3), human epidermal growth factor receptor 4 (HER4), human papillomavirus (HPV), mucin 1 (MUC1), prostate-specific antigen (PSA), PSMA, Brachyury, folate receptor alpha, WT1, p53, MAGE-A1, MAGE-A2, MAGE-A3, MAGE-A4, MAGE-A6, MAGE-A10, MAGE-A12, BAGE, DAM-6, -10, GAGE-1, -2, -8, GAGE-3, -4, -5, -6, -7B, NA88-A, NY-ESO-1, MART-1, MC1R, Gp100, Tyrosinase, TRP-1, TRP-2, ART-4, CAMEL, Cyp-B, BRCA1, BRACHYURY (TIVS7-2, polymorphism), BRACHYURY (IVS7 T/C polymorphism), T

BRACHYURY, T, hTERT, hTRT, iCE, MUC1 (VNTR polymorphism), MUC1c, MUC1n, MUC2, PRAME, P15, RU1, RU2, SART-1, SART-2, SART-3, AFP, 0-catenin/m, Caspase-8/m, CDK-4/m, ELF2M, GnT-V, G250, HSP70-2M, HST-2, KIAA0205, MUM-1, MUM-2, MUM-3, Myosin/m, RAGE, TRP-2/INT2, 707-AP, Annexin II, CDC27/m, TPI/mbcr-ab1, ETV6/AML, LDLR/FUT, Pm1/RARα, and TEL/AML1. Examples of the bispecific T cell activating antigen binding molecule are described in, for example, the following references: WO 00/006605 A2, U.S. Pat. No. 7,635,472 B2, WO 2005/040220 A1, WO 2008/119567 A2, WO 2010/037838 A2, WO 2013/026837 A1, WO 2013/026833 A1, US 2014/0308285 A1, WO 2014/144722 A2, WO 2014/151910 A1, WO 2015/048272 A1, and Sheridan, Nature Biotechnology, 34:1215-1217 (2016).

In one embodiment, effector memory γ9δ2 T cells are CD27(−) and CD45RA(−) cells. In a certain embodiment, effector memory γ9δ2 T cells do not express programmed death 1 (PD1 or PD-1) immune checkpoint molecules. In some embodiments, the T cells are autologous, allogenic or xenogenic to the subject in need thereof. In some preferred embodiments, the T cells are autologous or allogenic to the subject in need thereof.

Effector memory γ9δ2 T cells can be obtained from blood collected from a donor using any number of techniques known to the skilled artisan, such as Ficoll™ separation, and expanded ex vivo (Kondo et al., Cytotherapy, 10, 842-56 (2008)).

In one embodiment, the diseases that can be treated using the method of the invention include, but are not limited to, proliferative disorders (e.g. cancer), infectious diseases (e.g. bacterial infection, viral infection, yeast infection, fungal infection, protozoan infection, and parasite infection), or immune disorders (e.g. autoimmune diseases, allergies and immunodeficiency). In some embodiments, the disease is cancer. In other embodiments, the cancer is a solid tumor. In other embodiments, the cancer is a liquid tumor. In some embodiments, the cancer is a B cell cancer. In some embodiments, the B cell cancer is B cell lymphoma or B cell leukemia. In some embodiments, the B cell cancer is non-Hodgkin lymphoma, acute lymphoblastic leukemia or chronic lymphocytic leukemia. In some embodiments, the acute lymphoblastic leukemia is relapsed or refractory acute lymphoblastic leukemia.

Optionally, the method of the invention may comprise immunosuppressive therapies that suppress the immune system. Immunosuppressive therapy can help to alleviate, minimize, or eliminate transplant rejection in a recipient. For example, immunosuppressive therapy may comprise immuno-suppressive drugs. Immunosuppressive drugs that can be used before, during and/or after the administration of effector memory γ9δ2 T cells include, but are not limited to, MMF (mycophenolate mofetil (Cellcept)), ATG (anti-thymocyte globulin), anti-CD154 (CD4OL), anti-CD40 (2C10, ASKP1240, CCFZ533X2201), alemtuzumab (Campath), anti-CD20 (rituximab), anti-IL-6R antibody (tocilizumab, Actemra), anti-IL-6 antibody (sarilumab, olokizumab), CTLA4-Ig (Abatacept/Orencia), belatacept (LEA29Y), sirolimus (Rapimune), everolimus, tacrolimus (Prograf), daclizumab (Ze-napax), basiliximab (Simulect), infliximab (Remicade), cyclosporin, deoxyspergualin, soluble complement receptor 1, cobra venom factor, compstatin, anti C5 antibody (eculizumab/Soliris), methylprednisolone, FTY720, everolimus, leflunomide, anti-IL-2R-Ab, rapamycin, anti-CXCR3 antibody, anti-ICOS antibody, anti-OX40 antibody, and anti-CD122 antibody. Furthermore, one or more immunosuppressive agents/drugs can be used simultaneously or sequentially.

### Combination

The present invention further provides a combination comprising a therapeutically effective amount of a bispecific T cell activating antigen binding molecule and a therapeutically effective amount of effector memory γ9δ2 T cells, wherein the bispecific T cell activating antigen binding molecule can be administered to a subject simultaneously with, before or after the administration of the effector memory γ9δ2 T cells.

In one embodiment, the bispecific T cell activating antigen binding molecule and effector memory γ9δ2 T cells can together or individually dissolve or disperse in one or more pharmaceutically acceptable carriers. Pharmaceutically acceptable carriers include any and all solvents, buffers, dispersants, surfactants, antioxidants, preservatives (e.g. antibacterial agents, antifungal agents), isotonic agents, salts stabilizers, polymers, gels, binders, disintegration agents, lubricants, flavoring agents, such like materials and combinations thereof. In some embodiments, the bispecific T cell activating antigen binding molecule and effector memory γ9δ2 T cells are administered simultaneously, sequentially or intermittently.

In some embodiments, the bispecific T cell activating antigen binding molecule and effector memory γ9δ2 T cells can be administered intravenously, intraarterially, subcutaneously, intraperitoneally, intralesionally, intracranially, intramedullary, intraarticularly, intraprostatically, intrasplenically, intrarenally, intrapleurally, intratracheally, intranasally, intravitreally, intravaginally, intrarectally, intratumorally, intramuscularly, subconjunctivally, intravesicularly, mucosally, intrapericardially, intraumbilically, intraocularally, orally, topically, locally, by inhalation (e.g. aerosol inhalation), injection, infusion, continuous infusion, localized perfusion bathing target cells directly, via a catheter, via a lavage, in creams, in lipid compositions (e.g. liposomes), in microcapsules or by other method or any combination of the forgoing as would be known to one of ordinary skill in the art. In some embodiments, the bispecific T cell activating antigen binding molecule and effector memory γ9δ2 T cells are administered parenterally (e.g. subcutaneously, intradermically, intralesionally, intravenously, intraarterially, intramuscularly, intrathecally, or intraperitoneally) and, for example, simultaneously, sequentially or intermittently. Various dosing schedules including but not limited to single or multiple administrations over various time-points, bolus administration, and pulse infusion are contemplated herein.

For the treatment of disease, the appropriate dosage of bispecific T cell activating antigen binding molecule and effector memory γ9δ2 T cells will depend on the type of disease to be treated, the route of administration, the body weight of the patient, the type of bispecific T cell activating antigen binding molecule, the severity and course of the disease, previous or concurrent therapeutic interventions, the patient's clinical history, and the discretion of the attending physician. The practitioner responsible for administration will, in any event, determine the concentration of active ingredient(s) in a composition and appropriate dose(s) for the individual subject. For bispecific T cell activating antigen binding molecules, the daily dosage commonly used for administration to a patient is about 0.5 mg/kg to 1 mg/kg; and in general, the range of administration dosage for effector memory γ9δ2 T cells is between about 1×10⁴ and about 1×10¹¹ T cells, preferably between about 1×10⁶ and about 1×10⁹ T cells.

### Kit

In another embodiment, the invention provides a kit comprising substances suitable for treating the diseases as described above. The kit comprises a container and an instruction or package insert in or associated with the container. Suitable containers include, for example, bottles, vials, syringes, IV solution bags, etc. The containers may be formed from a variety of materials such as glass or plastic. The container holds a composition alone or combined with another composition effective for treating the diseases and may have a sterile access port (for example the container may be an intravenous solution bag or a vial having a stopper pierceable by a hypodermic injection needle). At least one active agent in the composition is the bispecific T cell activating antigen binding molecule or effector memory γ9δ2 T cells of the invention. The instruction or package insert indicates that the composition is used for treating the condition of choice. Moreover, the kit may comprise (a) a first container with a first composition contained therein, wherein the first composition comprises a bispecific T cell activating antigen binding molecule of the invention; and (b) a second container with a second composition contained therein, wherein the second composition comprises effector memory γ9δ2 T cells. The kit in this embodiment of the invention may further comprise a package insert indicating that the compositions can be used to treat a particular condition. Alternatively, or additionally, the kit may further comprise a third container comprising an immunosuppressive agent. It may further include other materials desirable from a commercial and user standpoint, including other buffers, diluents, filters, needles, and syringes.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### Isolation and Identification of γ9δ2 T Cells

For the purpose of the invention, effector memory γ9δ2 T cells that have the ability to kill blood cancer cells first need to be grown. γ9δ2 T cells can be identified by flow cytometry using anti-CD3 antibody, anti-γ9 antibody and anti-δ2 antibody. One of the characteristics of effector memory γ9δ2 T cells is the absence of CD27 and CD45RA molecules on the cell surface.

According to Kondo et al. (Cytotherapy, 10, 842-56 (2008)), after the monocytes of peripheral blood were stimulated and cultured with interleukin-2 (IL-2, 1000 U/ml) and Zoledronic acid (1 μM/ml) for 14 days, the ratio of δ2 cells in the culture was analyzed by flow cytometry using anti-δ2 antibody. The δ2 cells were then isolated using the TCRγ/δ+ T cell isolation Kit/human (Miltenyi Biotec). The isolated cells were analyzed by flow cytometry using anti-CD3 antibody, anti-γ9 antibody and anti-δ2 antibody to define their identity. The isolated cells were then assayed by anti-CD27 antibody, anti-CD45RA antibody and anti-PD-1 antibody to determine the expression of CD27, CD45RA and PD-1 molecules on γ9δ2 T cells.

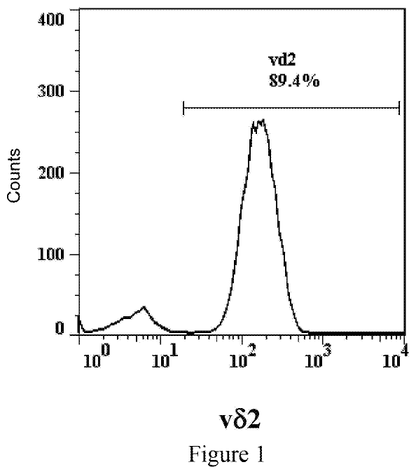

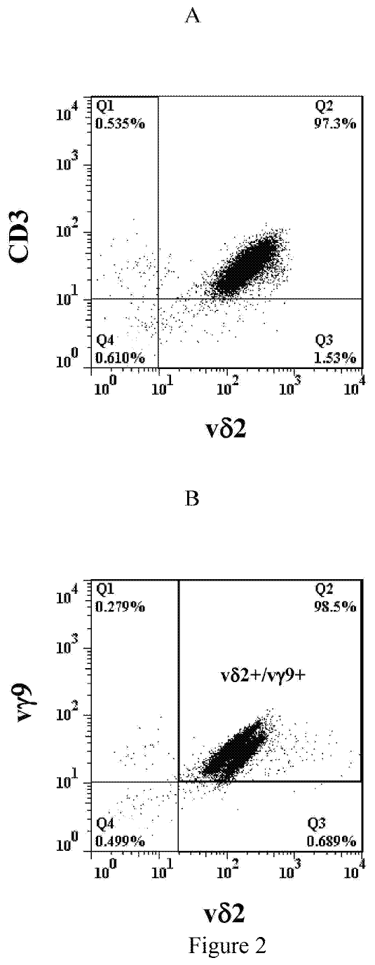

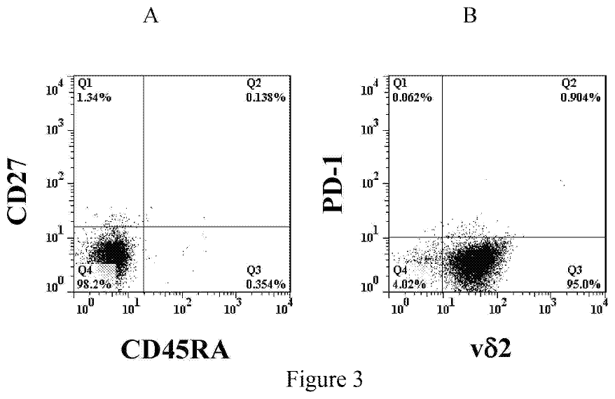

As shown in FIG. 1, more than 85% of the cells which were stimulated and cultured with IL-2 and Zoledronic acid for 14 days were δ2 cells. The δ2 cells isolated by the TCRγ/8+ T cell isolation Kit/human (Miltenyi Biotec) were all able to express CD3 molecule, and therefore, were a type of T cell. Furthermore, the proportion of γ9δ2 T cells was up to 98% (FIGS. 2A and B). FIG. 3A shows that these γ9δ2 T cells are all CD27(−) and CD45RA(−) cells, hence they are effector memory γ9δ2 T cells. FIG. 3B shows that these γ9δ2 T cells do not express PD-1 immune checkpoint molecules.

### Example 2

### In Vitro Assay for the Activity of γ9δ2 T Cells and BLINCYTO® (BiTE) on Killing Blood Cancer Cells

The experiment was carried out in accordance with the method disclosed in Sheehy et al. (J Immunol Methods, 249, 99-110 (2001)).

In this experiment, Raji, VAL and Daudi blood cancer cell lines expressing CD19 molecules were used as target cells, wherein the Raji and Daudi cell lines were CD19⁺ Burkett lymphoma cells, and VAL cell line was CD19⁺ ALL cells. The RPMI-8226 cell line was CD19⁻ multiple myeloma cells.

Raji, RPMI-8226, VAL and Daudi cell lines were independently stained with 5(6)-carboxyfluorescein diacetate succinimidyl ester (CFSE) and seeded in the wells of a culturing plate (5×10⁴/well). The γ9δ2 T cells (1×10⁶ cells/well) obtained from Example 1 and BLINCYTO® (15 ng/well) were added independently or together into each of the wells containing different cells. After being cultured for 6 hours, the viability of the cells was determined by counting CFSE+ cells through flow cytometry.

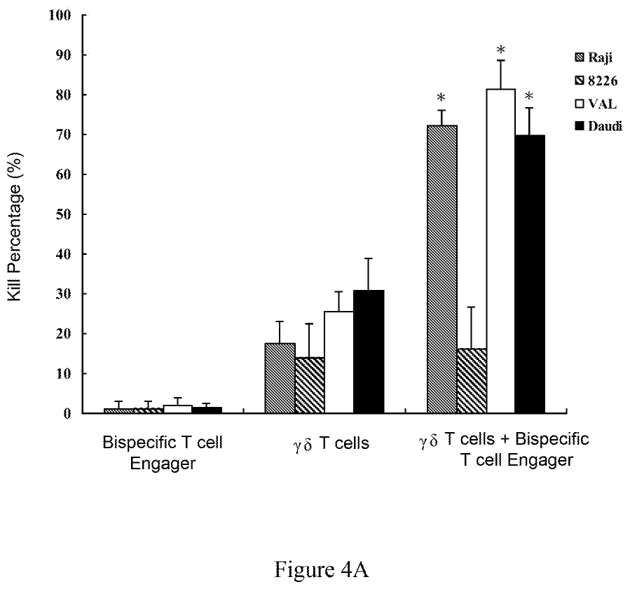

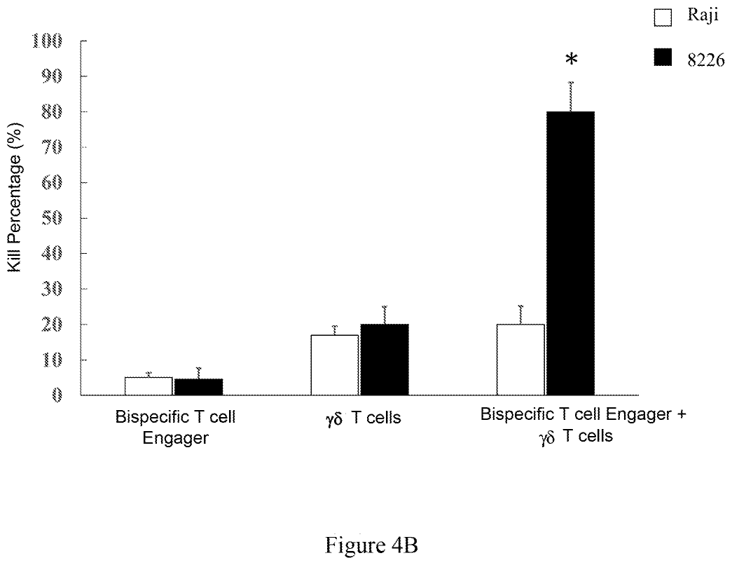

The results shown in FIG. 4A prove that γ9δ2 T cells not only have the ability to kill various blood cancer cells by themselves but also exhibit a synergistic effect in killing blood cancer cells when combined with BLINCYTO®. As compared with using γ9δ2 T cells or BLINCYTO® alone, the combination of γ9δ2 T cells and BLINCYTO® synergistically increased the effect of killing CD19⁺ blood cancer cells (Raji, VAL and Daudi blood cancer cell lines).

### In Vitro Assay for the Activity of γ9δ2 T Cells and BCMA-BiTE on Killing Blood Cancer Cells

The experiment was carried out in accordance with the method disclosed in Sheehy et al. (J Immunol Methods, 249, 99-110 (2001)). BCMA-BiTE was prepared according to WO2013/072415 A1.

In this experiment, RPMI-8226 blood cancer cell line expressing BCMA molecules was used as target cells, wherein the RPMI-8226 cell line was multiple myeloma cells.

Raji (BCMA⁻) and RPMI-8226 cell lines were independently stained with 5(6)-carboxyfluorescein diacetate succinimidyl ester (CFSE) and seeded in the wells of a culturing plate (5×10⁴ cells/well). The γ9δ2 T cells (1×10⁶ cells/well) obtained from Example 1 and BCMA-BiTE (15 μl/well) were added independently or together into each of the wells containing different cells. After being cultured for 6 hours, the viability of the cells was determined by counting CFSE+ cells through flow cytometry.

The results shown in FIG. 4B prove that γ9δ2 T cells not only have the ability to kill various blood cancer cells by themselves but also exhibit a synergistic effect in killing blood cancer cells when combined with BCMA-BiTE. As compared with using γ9δ2 T cells or BCMA-BiTE alone, the combination of γ9δ2 T cells and BCMA-BiTE synergistically increased the effect of killing RPMI-8226 blood cancer cells.

### Example 3

### In Vivo Assay for the Activity of γ9δ2 T Cells and BLINCYTO® on Killing Blood Cancer Cells in Immunodeficient NOG Mouse Model

This experiment was carried out in T cells-lacking immunodeficient NOG mice (with reference to Hipp et al., Leukemia, 31, 1-9 (2017), and Monjezi et al., Leukemia, 30, 1-9 (2016)).

First, the genes of luciferase and green fluorescent protein were transduced into VAL blood cancer cells (ACC-586) using lentiviral vector (Zhou et al., Blood, 120, 4334-4342 (2012).) The VAL blood cancer cells (5×10⁵ cells) that express luciferase and green fluorescent protein were implanted into NOG mice via tail vein injection.

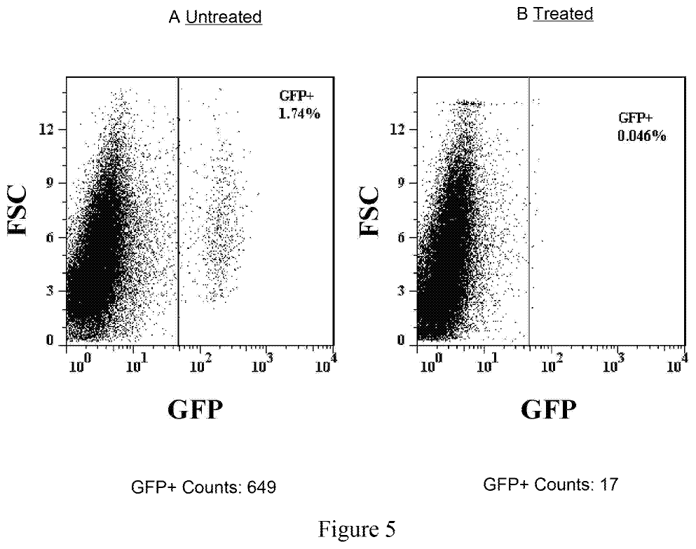

On day 4 after the implantation of VAL blood cancer cells, the γ9δ2 T cells obtained from Example 1 were implanted into the NOG mice via tail vein injection. The amount of γ9δ2 T cells implanted per injection was 20 fold the amount of VAL cells. The γ9δ2 T cells were implanted once every other day for a total of 7 implantations. In addition, on day 5 after the implantation of VAL blood cancer cells, BLINCYTO® was infused via tail vein for 14 consecutive days with a daily dose of 800 ng, which was divided into two injections with an 8-hour interval. The day after the completion of the treatment, the mice were sacrificed and the bone marrow was taken out to conduct flow cytometry to assay the activity of green fluorescence protein to determine the number of blood cancer cells. The mice of the control group did not have γ9δ2 T cells or BLINCYTO® implantations. The results showed that a great amount of VAL blood cancer cells still remained in the bone marrow of the mice of the control group (FIG. 5A). However, in the bone marrow of the mice treated with γ9δ2 T cells and BLINCYTO®, no significant VAL blood cancer cells were observed (FIG. 5B).

### Example 4

Survival Study of the Immunodeficient NOG Mice Treated with γ9δ2 T Cells and BLINCYTO®

In this experiment, T cells-lacking immunodeficient NOG mice were used for simulating patients (having very low number of T cells or lacking functional T cells (e.g. memory T cells)) having early recurrence after receiving bone marrow transplantation.

Furthermore, in this experiment, peripheral blood mononuclear cells (PBMC) containing memory T cells were transplanted into NOG mice for simulating patients having recurrence without receiving bone marrow transplantation.

According to the method described in Example 3, the VAL blood cancer cells (5×10⁵ cells) expressing luciferase and green fluorescent protein were implanted into two groups of NOG mice via tail vein injection. The NOG mice without PBMC transplantation were treated according to the treatment described in Example 3. For the NOG mice with PBMC transplantation, PBMCs obtained from peripheral blood were first treated with Cyclophosphamide, Vincristine, and Doxorubicin Hydrochloride for 3 days to mimic that of ALL patients treated by chemotherapy and then implanted via tail vein injection on day 4 after the implantation of VAL blood cancer cells. The implanted cell number of PBMC was 20 fold that of VAL cells, and the implantation was performed once every three days. The treatment of γ9δ2 T cells and BLINCYTO® to the NOG mice with PBMC transplantation was carried out as described in Example 3.

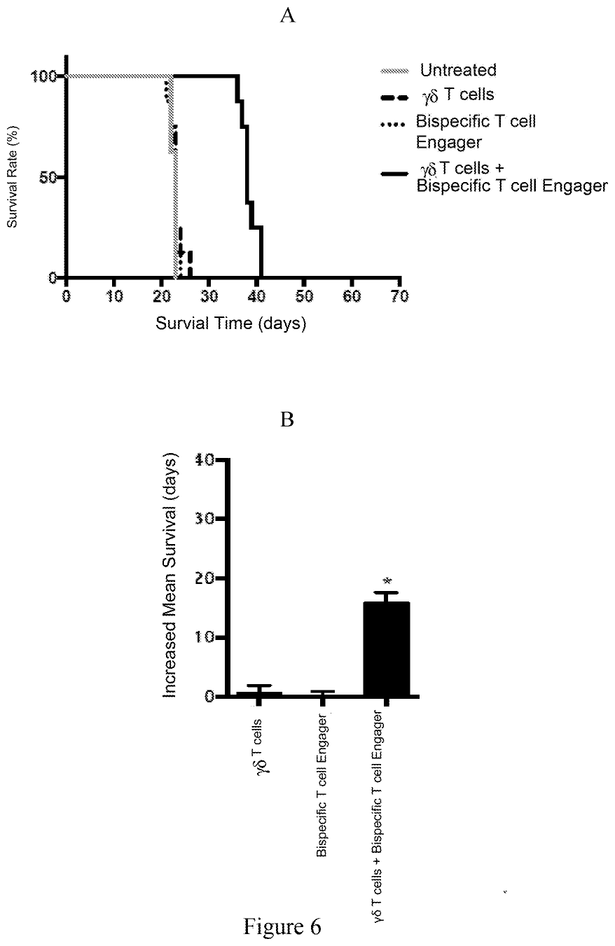

FIGS. 6A and B provide the results of survival curves and relative days of survival (p<0.01) of the VAL blood cancer cells in the NOG mice (without PBMC transplantation) simulating patients having early recurrence after receiving bone marrow transplantation. The results show that the average survival days of the mice treated with γ9δ2 T cells or BLINCYTO® alone are 1.5 days longer than those of the mice without the treatment with γ9δ2 T cells and BLINCYTO®; and that the average survival days of the mice treated with γ9δ2 T cells in combination with BLINCYTO® are about 15 days longer. Accordingly, the combination therapy of γ9δ2 T cells and BLINCYTO® can synergistically extend the survival of NOG mice as compared with the treatment with γ9δ2 T cells or BLINCYTO® alone.

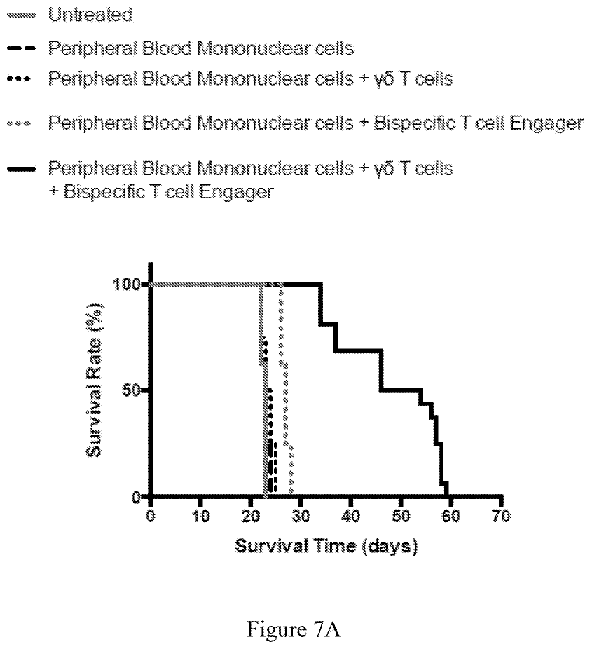

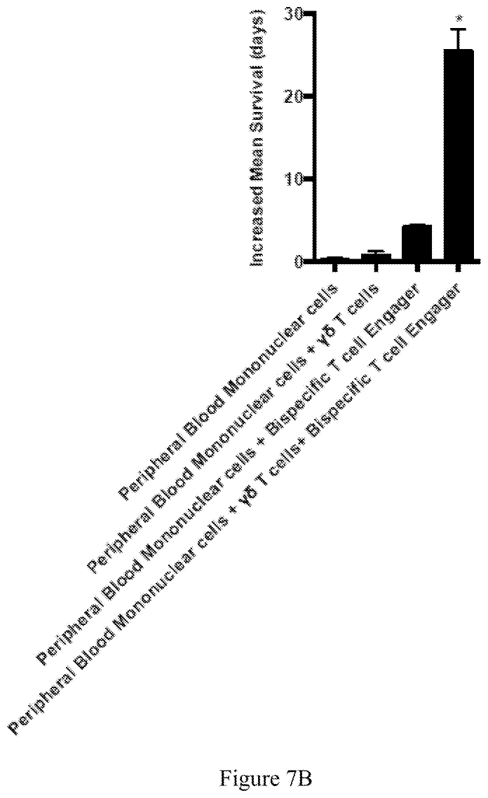

FIGS. 7A and B show the survival curves and relative days of survival (p<0.001) of the VAL blood cancer cells in NOG mice (with PBMC transplantation) simulating patients having early recurrence without receiving bone marrow transplantation. The results show that the average survival days of the NOG mice with PBMC transplantation treated with γ9δ2 T cells alone are 1.5 days longer than those of the untreated mice; and that the average survival days of the mice treated with BLINCYTO® alone are about 5 days longer than those of untreated. However, for the mice treated with the combination of γ9δ2 T cells and BLINCYTO®, the average survival days may be up to about 25.5 days longer than those of untreated, which is about 3.9 times to the total survival days of the mice treated with γ9δ2 T cells or BLINCYTO® alone. Accordingly, the combination therapy of γ9δ2 T cells and BLINCYTO® can synergistically extend the survival of NOG mice with PBMC transplantation as compared with the treatment with γ9δ2 T cells or BLINCYTO® alone.

### Example 5

### Combination Therapy Study of 79δ2 T Cells and BLINCYTO® for Extramedual Diseases in Immunodeficient NOG Mice

BLINCYTO® has been marketed for more than two years in the United States. When analyzing the effect of BLINCYTO® in clinical application, Aldoss et al. (AM J Hematol., 92, 858-865 (2017)) found that if the patients had extramedual diseases prior to the treatment with BLINCYTO®, the clinical therapeutic effect of BLINCYTO® would be poor. In addition, of the patients who had recurrence after receiving the treatment with BLINCYTO®, up to 40% had extramedual diseases.

In this experiment, the VAL blood cancer cells (5×10⁵ cells) having the luciferase and green fluorescent protein dual genes were implanted into NOG mice via tail vein injection. Starting from day 7, γ9δ2 T cells and BLINCYTO® were implanted into the NOG mice according to the method described in Example 3 but the duration of treatment was reduced to 6 days. Control group was administered with BLINCYTO® only starting from day 7. On the day the treatment started and at different time points after the treatment began, the NOG mice were assayed by IVIS to measure the intensity of luciferase activity in the abdomen and chest as the indication of extramedular growth of cancer cells.

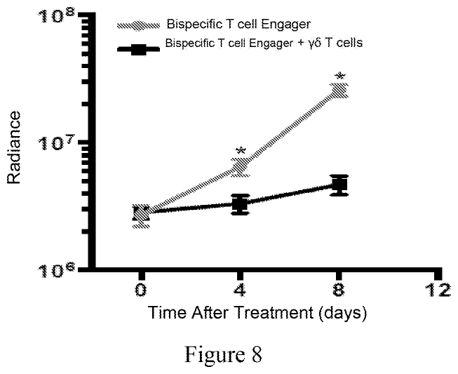

The results are shown in FIG. 8. The combination therapy of γ9δ2 T cells and BLINCYTO® can significantly reduce the occurrence of extramedular growth of cancer cells as compared with the treatment with BLINCYTO® alone (p<0.01).

1. A combination, comprising a therapeutically effective amount of a bispecific T cell activating antigen binding molecule and a therapeutically effective amount of CD27(−) and CD45RA(−) effector memory γ9δ2 T cells,
wherein the bispecific T cell activating antigen binding molecule comprises:
(a) a first antigen binding moiety specifically binding a first antigen, wherein the first antigen is CD3, which activates T cells, and
(b) a second antigen binding moiety specifically binding a second antigen, wherein the second antigen is selected from the group consisting of a tumor cell neo-antigen, a tumor neo-epitope, a tumor-specific antigen, a tumor associated antigen, a tissue-specific antigen, a bacterial antigen, a viral antigen, a yeast antigen, a fungal antigen, a protozoan antigen, and a parasite antigen, and
wherein the effector memory γ9δ2 T cells do not express programmed death-1 (PD-1).

2. (canceled)

3. The combination according to claim 1, wherein the second antigen is selected from the group consisting of CD19, CD20, CD22, CD31, CD32B, CD33, CD34, CD40, CD117, CD123, fibroblast-activating protein (FAP), fibroblast growth factor receptor 1 (FGFR1), B-cell maturation antigen (BCMA), carcinoembryonic antigen (CEA), endothelial growth factor receptor, glycoprotein A33 antigen (gpA33), human epidermal growth factor receptor 1 (HER1), human epidermal growth factor receptor 2 (HER2/neu), human epidermal growth factor receptor 3 (HER3), human epidermal growth factor receptor 4 (HER4), human papillomavirus (HPV), mucin 1 (MUC1), prostate-specific antigen (PSA), PSMA, Brachyury, folate receptor alpha, WT1, p53, MAGE-A1, MAGE-A2, MAGE-A3, MAGE-A4, MAGE-A6, MAGE-A10, MAGE-A12, BAGE, DAM-6, -10, GAGE-1, -2, -8, GAGE-3, -4, -5, -6, -7B, NA88-A, NY-ESO-1, MART-1, MC1R, Gp100, Tyrosinase, TRP-1, TRP-2, ART-4, CAMEL, Cyp-B, BRCA1, BRACHYURY (TIVS7-2, polymorphism), BRACHYURY (IVS7 T/C polymorphism), T BRACHYURY, T, hTERT, hTRT, iCE, MUC1 (VNTR polymorphism), MUC1c, MUC1n, MUC2, PRAME, P15, RU1, RU2, SART-1, SART-2, SART-3, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, TRP-2/INT2, 707-AP, Annexin II, CDC27/m, TPI/mbcr-ab1, ETV6/AML, LDLR/FUT, Pm1/RARα, and TEL/AML1.

4. (canceled)

5. The combination according to claim 1, wherein the effector memory γ9δ2 T cells are expanded ex vivo.

6. The combination according to claim 1, wherein the effector memory γ9δ2 T cells are autologous or allogenic to a subject in need thereof.

7. The combination according to claim 1, wherein the bispecific T cell activating antigen binding molecule is formulated into a pharmaceutical composition.

8. The combination according to claim, wherein the effector memory γ9δ2 T cells are formulated into a pharmaceutical composition.

9. A method for treating a disease in a subject in need thereof, comprising simultaneously, sequentially or intermittently administering to the subject a therapeutically effective amount of CD27(−) and CD45RA(−) effector memory γ9δ2 T cells and a therapeutically effective amount of a bispecific T cell activating antigen binding molecule,
wherein the bispecific T cell activating antigen binding molecule comprises:
(a) a first antigen binding moiety specifically binding a first antigen, wherein the first antigen is CD3, which activates T cells; and
(b) a second antigen binding moiety specifically binding a second antigen, wherein the second antigen is selected from the group consisting of a tumor cell neo-antigen, a tumor neo-epitope, a tumor-specific antigen, a tumor associated antigen, a tissue-specific antigen, a bacterial antigen, a viral antigen, a yeast antigen, a final antigen, a protozoan antigen, and a parasite antigen, and
wherein the effector memory γ9δ2 T cells do not express programmed death-1 (PD-1).

10. (canceled)

11. The method according to claim 9, wherein the effector memory γ9δ2 T cells are expanded ex vivo.

12. The method according to claim 9, wherein the effector memory γ9δ2 T cells are autologous or allogenic to the subject in need thereof.

13. (canceled)

14. The method according to claim 9, wherein the second antigen is selected from the group consisting of CD19, CD20, CD22, CD31, CD32B, CD33, CD34, CD40, CD117, CD123, fibroblast-activating protein (FAP), fibroblast growth factor receptor 1 (FGFR1), B-cell maturation antigen (BCMA), carcinoembryonic antigen (CEA), endothelial growth factor receptor, glycoprotein A33 antigen (gpA33), human epidermal growth factor receptor 1 (HER1), human epidermal growth factor receptor 2 (HER2/neu), human epidermal growth factor receptor 3 (HER3), human epidermal growth factor receptor 4 (HER4), human papillomavirus (HPV), mucin 1 (MUC1), prostate-specific antigen (PSA), PSMA, Brachyury, folate receptor alpha, WT1, p53, MAGE-A1, MAGE-A2, MAGE-A3, MAGE-A4, MAGE-A6, MAGE-A10, MAGE-A12, BAGE, DAM-6, -10, GAGE-1, -2, -8, GAGE-3, -4, -5, -6, -7B, NA88-A, NY-ESO-1, MART-1, MC1R, Gp100, Tyrosinase, TRP-1, TRP-2, ART-4, CAMEL, Cyp-B, BRCA1, BRACHYURY (TIVS7-2, polymorphism), BRACHYURY (IVS7 T/C polymorphism), T BRACHYURY, T, hTERT, hTRT, iCE, MUC1 (VNTR polymorphism), MUC1c, MUC1n, MUC2, PRAME, P15, RU1, RU2, SART-1, SART-2, SART-3, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, TRP-2/INT2, 707-AP, Annexin II, CDC27/m, TPI/mbcr-ab1, ETV6/AML, LDLR/FUT, Pm1/RARα, and TEL/AML1.

15. The method according to claim 9, wherein the disease is cancer.

16. The method according to claim 15, wherein the cancer is a solid tumor or a liquid tumor.

17. The method according to claim 16, wherein the liquid tumor is non-Hodgkin lymphoma or acute lymphoblastic leukemia or chronic lymphocytic leukemia.

18. The method according to claim 17, wherein the acute lymphoblastic leukemia is relapsed or refractory acute lymphoblastic leukemia.

19. (canceled)

20. (canceled)

21. (canceled)

22. (canceled)

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. A kit, comprising a bispecific T cell activating antigen binding molecule, CD27(−) and CD45RA(−) effector memory γ9δ2 T cells, and a package insert indicating the disease to be treated in a subject in need thereof,
wherein the bispecific T cell activating antigen binding molecule comprises:
(a) a first antigen binding moiety specifically binding a first antigen, wherein the first antigen is CD3, which activates T cells; and
(b) a second antigen binding moiety specifically binding a second antigen, wherein the second antigen is selected from the group consisting of a tumor cell neo-antigen a tumor neo-epitope, a tumor-specific antigen, a tumor associated antigen, a tissue-specific antigen, a bacterial antigen, a viral antigen, a yeast antigen, a fungal antigen, a protozoan antigen, and a parasite antigen, and
wherein the effector memory γ9δ2 T cells do not express programmed death-1 (PD-1).

36. (canceled)

37. The kit according to claim 35, wherein the second antigen is selected from the group consisting of CD19, CD20, CD22, CD31, CD32B, CD33, CD34, CD40, CD117, CD123, fibroblast-activating protein (FAP), fibroblast growth factor receptor 1 (FGFR1), B-cell maturation antigen (BCMA), carcinoembryonic antigen (CEA), endothelial growth factor receptor, glycoprotein A33 antigen (gpA33), human epidermal growth factor receptor 1 (HER1), human epidermal growth factor receptor 2 (HER2/neu), human epidermal growth factor receptor 3 (HER3), human epidermal growth factor receptor 4 (HER4), human papillomavirus (HPV), mucin 1 (MUC1), prostate-specific antigen (PSA), PSMA, Brachyury, folate receptor alpha, WT1, p53, MAGE-A1, MAGE-A2, MAGE-A3, MAGE-A4, MAGE-A6, MAGE-A10, MAGE-A12, BAGE, DAM-6, -10, GAGE-1, -2, -8, GAGE-3, -4, -5, -6, -7B, NA88-A, NY-ESO-1, MART-1, MC1R, Gp100, Tyrosinase, TRP-1, TRP-2, ART-4, CAMEL, Cyp-B, BRCA1, BRACHYURY (TIVS7-2, polymorphism), BRACHYURY (IVS7 T/C polymorphism), T BRACHYURY, T, hTERT, hTRT, iCE, MUC1 (VNTR polymorphism), MUC1c, MUC1n, MUC2, PRAME, P15, RU1, RU2, SART-1, SART-2, SART-3, AFP, β-catenin/m, Caspase-8/m, CDK-4/m, ELF2M, GnT-V, G250, HSP70-2M, HST-2, KIAA0205, MUM-1, MUM-2, MUM-3, Myosin/m, RAGE, TRP-2/INT2, 707-AP, Annexin II, CDC27/m, TPI/mbcr-ab1, ETV6/AML, LDLR/FUT, Pm1/RARα, and TEL/AML1.

38. (canceled)

39. The kit according to claim 35, wherein the effector memory γ9δ2 T cells are expanded ex vivo.

40. The kit according to claim 35, wherein the effector memory γ9δ2 T cells are autologous or allogenic to the subject in need thereof.

41. The kit according to claim 35, wherein the bispecific T cell activating antigen binding molecule is formulated into a first pharmaceutical composition.

42. The kit according to claim 35, wherein the effector memory γ9δ2 T cells are formulated into a second pharmaceutical composition.

43. (canceled)

44. (canceled)

45. (canceled)

46. (canceled)
